(19) United States
(12) Patent Application Publication
(10) Pub. No.: US 2017/0049891 A1
(43) Pub. Date: Feb. 23, 2017

(54) 5-AMINOLEVULINIC ACID CONJUGATED QUANTUM DOT NANOPARTICLE

(71) Applicant: Nanoco Technologies Ltd., Manchester (GB)

(72) Inventor: Imad Naasani, Manchester (GB)

(21) Appl. No.: 15/235,362

(22) Filed: Aug. 12, 2016

Related U.S. Application Data
(60) Provisional application No. 62/205,998, filed on Aug. 17, 2015.

(51) Int. Cl. A61K 41/00; A61K 47/48; A61M 5/00; G01N 33/58; A61B 5/00; A61K 9/50; A61K 49/00

(52) U.S. Cl. CPC A61K 41/0061; A61K 9/501; A61K 47/48246; A61K 47/48861; A61K 49/005; A61K 49/0036; G01N 33/587; A61B 5/0071; A61M 5/007; B82Y 15/00

(57) ABSTRACT

A 5-aminolevulinic acid conjugated quantum dot nanoparticle is useful for treating cancer by administering the 5-aminolevulinic acid conjugated quantum dot nanoparticle in photodynamic therapy as a precursor of both a fluorescence label and a photosensitizer.

### CROSS-REFERENCE TO RELATED APPLICATIONS

This application claims the benefit of U.S. Provisional Application No. 62/205,998 filed on Aug. 17, 2015.

### STATEMENT REGARDING FEDERALLY SPONSORED RESEARCH OR DEVELOPMENT

Not Applicable

### BACKGROUND OF THE INVENTION

1. Field of the Invention

The present invention relates to 5-aminolevulinic acids and their derivatives conjugated to quantum dot nanoparticles, and methods of preparing 5-aminolevulinic acids and their derivatives conjugated to quantum dot nanoparticles. The present invention also relates to methods of treating cancer by administering 5-aminolevulinic acids and their derivatives conjugated to quantum dot nanoparticle in photodynamic therapy as a precursor of both a fluorescence label and a photosensitizer.

2. Description of the Related Art Including Information Disclosed Under 37 CFR 1.97 and 1.98.

Photodynamic therapy (PDT) is a treatment that uses a photosensitive drug, called a photosensitizer (PS), along with light to kill cancer cells. The drugs only work after they have been activated by light. Upon irradiation with appropriate light, the photosensitizer produces reactive oxygen species (ROS) for the destruction of the neoplastic tissue.

5-Aminolevulinic acid (5-ALA) is an approved PS for PDT and is widely used. Derivatives and analogs of 5-ALA have also been proposed as a PS for PDT; specifically, ester derivatives of 5-ALA as disclosed in WO 2002009690, incorporated by reference herein in its entirety. 5-ALA and its derivatives and analogs are a prodrug, and once internalized into tumor cells, undergoes conversion to the natural photosensitizer photoporphyrin IX (PpIX). Unlike exogenously administered PSs, such as PHOTOFRIN® (porfimer sodium) [CONCORDIA LABORATORIES INC. ST. MICHAEL BARBADOS BB11005], the photodynamically inactive, non-selective and non-toxic 5-ALA is intracellularly metabolized to the photodynamically active and fluorescent PpIX. Subsequent illumination of the tumor site with light, for example, blue light, activates PpIX, triggers the oxidative damage and induces cytotoxicity.

However, 5-ALA is a polar molecule. The zwitterionic nature and hydrophilicity of 5-ALA greatly limit its penetration through tissues, such as intact skin, nodular skin lesions and through cell membranes, leading to a slow cellular uptake and an inconsistent accumulation of PpIX in tumor cells. Thus, 5-ALA penetration through the cell membrane and targeted delivery to tumor cells are challenges in improving the efficacy and specificity of PDT.

Additionally, 5-ALA may also be a marker in fluorescence-guided surgeries of cancers such as gliomas and melanomas. The above-discussed limitations render 5-ALA's ability as a labeling agent unsatisfactory for this application as well.

### SUMMARY OF THE INVENTION

There has been substantial interest in the preparation and characterization of particles in the range 2-100 nm, often referred to as quantum dots as compound semiconductors, in phototherapy, displays, lighting, solar energy and bio-imaging.

U.S. Pat. No. 7,588,828 (filed Sep. 10, 2007 and issued Sep. 15, 2009), U.S. Pat. No. 7,803,423 (filed Apr. 27, 2005 and issued Sep. 28, 2010), U.S. Pat. No. 7,867,556, U.S. Pat. No. 7,985,446 (filed Aug. 11, 2010 and issued Jul. 26, 2011), U.S. Pat. No. 8,062,703 (filed Aug. 10, 2010 and issued Nov. 22, 2011), Applicant's commonly owned U.S. application Ser. No. 14/207,084, Applicant's commonly owned U.S. application Ser. No. 14/212,702, and Applicant's commonly owned U.S. application Ser. No. 14/208,311, the entire contents of which are hereby incorporated by reference in their entireties, describe methods of producing large volumes of high-quality, monodisperse QDs. QD precursors are provided in the presence of a molecular cluster compound under conditions whereby the integrity of the molecular cluster is maintained and acts as a well-defined prefabricated seed or template to provide nucleation centers that react with the chemical precursors to produce high quality nanoparticles on a sufficiently large scale for industrial application.

QD particles may be functionalized with organic end groups for further chemical manipulation. One example is a passivating layer. In the process of preparing QDs, the coordination about the final inorganic surface atoms in any nanoparticle may be incomplete, with highly reactive non-fully coordinated atomic “dangling bonds” on the surface of the particle, which may lead to particle agglomeration. To overcome this problem, an organic passivating layer may be employed to cap the bare surface atoms with protective organic groups. The passivating layer provides organic functional groups through which chemical linkage to other materials are possible.

The present invention provides a conjugate comprising 5-ALA, its derivatives, and its analogs conjugated to a nanoparticle conjugate. In one embodiment, 5-ALA is bonded to a nanoparticle. The 5-ALA may be conjugated with the nanoparticle either covalently, physically, ion pairing, or Van der Waals' interactions. The bond may be formed by an amide, ester, thioester, or thiol anchoring group directly on the inorganic surface of the quantum dot nanoparticle, or on the organic corona layer that is used to render the nanoparticles water soluble and biocompatible.

In one embodiment, the 5-ALA-nanoparticle conjugate comprises: a molecular cluster compound, a core semiconductor material, and an outer layer, wherein the outer layer comprises R, wherein R is

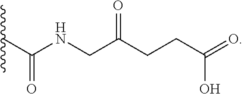 [SMILES: *C(=O)NCC(=O)CCC(=O)O]

In another embodiment, the nanoparticle is an alloyed quantum dot. Unlike core-shell structured nanoparticles, alloyed nanoparticles do not have a defined core-shell configuration and possess a graded band gap.

Embodiments also provide methods of preparing a 5-ALA-nanoparticle conjugate described above, comprising the steps: 1) coupling a nanoparticle with 5-ALA to give crude 5-ALA-nanoparticle conjugate, wherein the nanoparticle comprises outer layer having a carboxyl group; 2) purifying the crude 5-ALA-nanoparticle conjugate; and 3) isolating the 5-ALA-nanoparticle conjugate.

Embodiments provide systems for a fluorescence labeling agent and a photosensitizer, comprising a 5-ALA-nanoparticle conjugate comprising: quantum dot having an outer layer comprises R, wherein R is

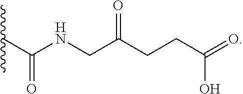 [SMILES: *C(=O)NCC(=O)CCC(=O)O]

Embodiments provide methods of treating cancer, comprising the step of administering a 5-ALA-nanoparticle conjugate in photodynamic therapy as a precursor of both a fluorescence label and a photosensitizer, and subsequently irradiating the photosensitizer.

Embodiments provide methods of inducing cell apoptosis comprising the steps of administering a 5-ALA-nanoparticle conjugate in photodynamic therapy as a precursor of both a fluorescence label and a photosensitizer, and subsequently irradiating the photosensitizer.

Embodiments provide further conjugating a 5-ALA-nanoparticle conjugate with a tissue-specific ligand, such as, for example, a peptide capable of targeting specific tissue(s) for uptake of the 5-ALA-nanoparticle conjugate. An example of such a peptide is an antibody capable of targeting cancerous cells and neoplastic tissues including tumors. Examples of targeted cancers include cancer of the prostate, breast, colon, skin, cervix, bladder, lung, and stomach. The peptide capable of targeting specific tissue(s) may be conjugated with the nanoparticle either covalently, physically, by ion pairing, or Van der Waals' interactions. The bond may be formed by an amide, ester, thioester, or thiol anchoring group directly on the inorganic surface of the quantum dot nanoparticle, or on the organic corona layer that is used to render the nanoparticles water soluble and biocompatible.

Embodiments include administering a 5-ALA-nanoparticle conjugate subcutaneously, intravenously, intramuscular, topically, and orally. Examples include bolus injections or IV infusions.

Embodiments also include methods of diagnosing cancer comprising the steps of administering a 5-ALA-nanoparticle conjugate in photodynamic diagnosis as a precursor of both a fluorescence label and a photosensitizer, 5-ALA disassociating from the nanoparticle and forms PpIX, and exciting a disassociated nanoparticle to emit blue light of 375-475 nm, activating the fluorescent properties of PpIX, and imaging the fluorescence to detect cancer.

Embodiments also include methods of surgical excision of tumor cells comprising the steps of administering a 5-ALA-nanoparticle conjugate in photodynamic diagnosis as a precursor of both a fluorescence label and a photosensitizer, 5-ALA disassociating from the nanoparticle and forms PpIX, and exciting a disassociated nanoparticle to emit blue light of 375-475 nm, and activating the fluorescent properties of PpIX thereby allowing detection and removal of the tumor cells.

### BRIEF DESCRIPTION OF THE SEVERAL VIEWS OF THE DRAWING(S)

FIG. 1 is a schematic diagram of a process of preparing a 5-ALA-nanoparticle conjugate.

FIG. 2 illustrates the conjugation with 5-ALA of a nanoparticle (represented by the filled circle) having surface-bound ligands attached thereto. In this representative illustration, X=a surface binding ligand (thiol, amine, phosphine, phosphine oxide, carboxylic acid, etc.), Y=a linking group (hydrocarbon chain comprising one or more of alkyls, alkenyls, alkynyls; polymers such as PEG, PPO, PEO, silicone rubber, polyethylene, acrylic resins, polyurethane, polypropylene, and polymethylmethacrylate; copolymers; block copolymers, etc.), and Z=a carboxylic acid, ester, acyl chloride, acid anhydride, or aldehyde.

FIG. 3 illustrates a metabolic pathway from the 5-ALA-nanoparticle conjugate of FIG. 2 to the photosensitizer protoporphyrin IX (PpIX or PROTO).

### DETAILED DESCRIPTION OF THE INVENTION

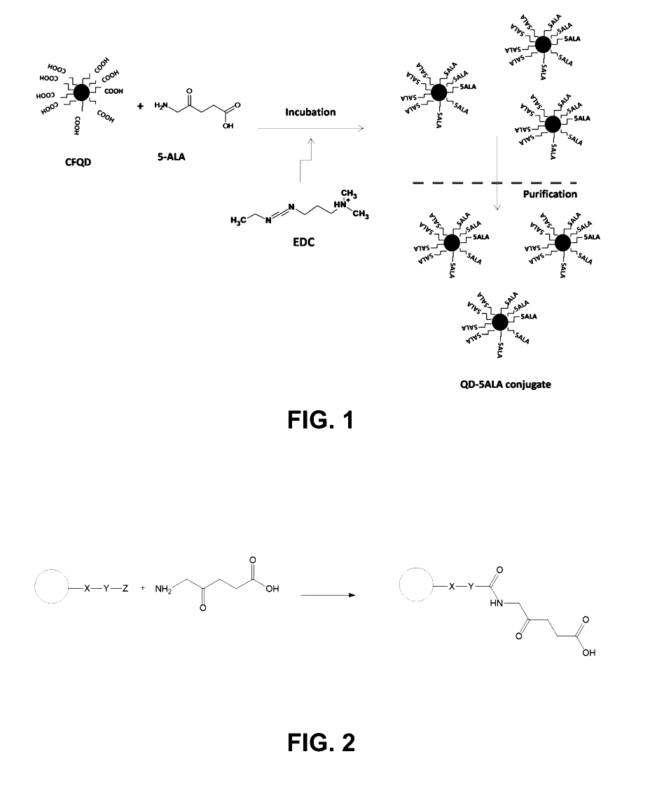

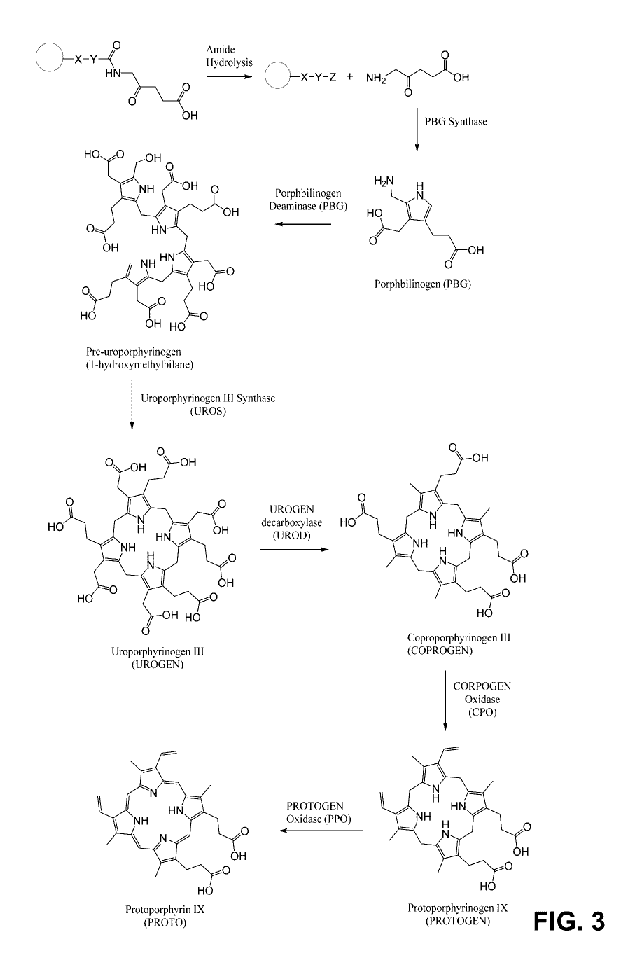

In FIG. 1, a 5-ALA-nanoparticle conjugate is provided by reacting a nanoparticle with 5-ALA. As an example, the nanoparticle comprises a molecular cluster compound, a core semiconductor material, and an outer layer. The outer layer comprises a carboxyl group with which 5-ALA reacts to form a linkage. It should be understood that derivatives and analogs of 5-ALA could be used either alone or in combination. It should also be understood that an alloyed nanoparticle may be also be used. In addition, a combination of core-shell nanoparticles and alloyed nanoparticles may be used.

Derivatives of 5-ALA include, but are not limited to:

5-ALA n-alkyl esters
5-ALA methyl ester (methylaminolevulinate, Trade name METVIV™)
5-ALA ethyl ester
5-ALA propyl ester
5-ALA butyl ester
5-ALA pentyl ester
5-ALA hexyl ester (hexylaminolevulinate, Trade name HEXVIX™)
5-ALA octyl ester

As well as:
5-ALA (hydroxymethyl)tetrahydrofuranyl ester; and,
5-ALA polyethylene glycol derivatives

Plus salts such as:
5-ALA.HCl

The types of core-shell nanoparticles include but are not limited to core material comprising the following types:

IIA-VIB (2-16) material, consisting of a first element from Group 2 of the periodic table and a second element from Group 16 of the periodic table and also including ternary and quaternary materials and doped materials. Nanoparticle material include but are not restricted to: MgS, MgSe, MgTe, CaS, CaSe, CaTe, SrS, SrSe, SrTe, BaS, BaSe, BaTe.

IIB-VIB (12-16) material consisting of a first element from Group 12 of the periodic table and a second element from Group 16 of the periodic table and also including ternary and quaternary materials and doped materials. Nanoparticle material includes but are not restricted to: ZnS, ZnSe, ZnTe, CdS, CdSe, CdTe, HgS, HgSe, HgTe.

II-V material consisting of a first element from Group 12 of the periodic table and a second element from Group 15 of the periodic table and also including ternary and quaternary materials and doped materials. Nanoparticle material include but is not restricted to: Zn₃P₂, Zn₃As₂, Cd₃P₂, Cd₃As₂, Cd₃N₂, Zn₃N₂.

III-V material consisting of a first element from Group 13 of the periodic table and a second element from Group 15 of the periodic table and also including ternary and quaternary materials and doped materials. Nanoparticle material include but is not restricted to: BP, AlP, AlAs, AlSb; GaN, GaP, GaAs, GaSb; InN, InP, InAs, InSb, AlN, BN.

III-IV material consisting of a first element from Group 13 of the periodic table and a second element from Group 14 of the periodic table and also including ternary and quaternary materials and doped materials. Nanoparticle material include but is not restricted to: B₄C, Al₄C₃, Ga₄C.

III-VI material consisting of a first element from Group 13 of the periodic table and a second element from Group 16 of the periodic table and also including ternary and quaternary materials. Nanoparticle material include but is not restricted to: Al₂S₃, Al₂Se₃, Al₂Te₃, Ga₂S₃, Ga₂Se₃, GeTe; In₂S₃, In₂Se₃, Ga₂Te₃, In₂Te₃, InTe.

IV-VI material consisting of a first element from Group 14 of the periodic table and a second element from Group 16 of the periodic table, and also including ternary and quaternary materials and doped materials. Nanoparticle material include but is not restricted to: PbS, PbSe, PbTe, Sb₂Te₃, SnS, SnSe, SnTe.

Nanoparticle material consisting of a first element from any Group in the transition metal of the periodic table, and a second element from any group of the d-block elements of the periodic table and also including ternary and quaternary materials and doped materials. Nanoparticle material include but is not restricted to: NiS, CrS, CuInS₂.

The term “doped nanoparticle” for the purposes of this specification and its claims refers to nanoparticles of the above and a dopant comprising one or more main group or rare earth elements. This most often is a transition metal or rare earth element, such as but not limited to zinc sulfide with manganese, such as ZnS nanoparticles doped with Mn⁺.

In one embodiment, cadmium-free nanoparticles are preferred.

In an embodiment, the nanoparticle includes a first layer including a first semiconductor material provided on the nanoparticle core. A second layer including a second semiconductor material may be provided on the first layer.

Standard conjugation chemistry may be used for conjugation. For example, a method preparing a 5-ALA-nanoparticle conjugate may include the steps of providing a nanoparticle, providing a coupling agent, providing 5-ALA, 5-ALA derivatives (such as, for example, its ester derivatives), 5-ALA analogs, incubating the mixture to form a crude 5-ALA-nanoparticle conjugate. The crude 5-ALA-nanoparticle conjugate may then be purified and isolated to obtain a 5-ALA-nanoparticle conjugate.

The incubations conditions may be chosen to allow for formation of either an amide or an ester. It should be understood that other bonds may be formed (e.g., both covalent and non-covalent). In one embodiment, 5-ALA is bonded to a nanoparticle. The 5-ALA may be conjugated with the nanoparticle either covalently, physically, ion pairing, or Van der Waals' interactions. The bond may be formed by an amide, ester, thioester, or thiol anchoring group directly on the inorganic surface of the quantum dot nanoparticle, or on the organic corona layer that is used to render the nanoparticles water soluble and biocompatible.

Standard incubation conditions for coupling may be employed. For example, the coupling conditions may be a solution in the range of 0.5 to 4 hours. The temperature range of the coupling conditions may be in the range of 100° C. to 200° C. The coupling conditions may be constant or varied during the reaction. For example, the reaction conditions may be 130° C. for one hour then raised to 140° C. for three hours.

Linkers may be used to form an amide or an ester group between the carboxyl functions on the nanoparticles and either the carboxyl or the amine end groups on the 5-ALA. Linkers or coupling agents may include benzotriazolyloxy-tris(dimethylamino) phosphonium Hexafluorophosphate (BOP) and carbodiim ides such as dicyclohexylcarbodiimide (DCC), diisopropylcarbodiimide (DIC), and 1-(3-dimethyl-aminopropyl)-3-ethylcarbodiimide hydrochloride (EDC). EDC is a preferred carbodiimide to use as the amide coupling agent.

In an example, the quantum dot nanoparticles bearing a carboxyl end group and 5-ALA may be mixed in a solvent. A coupling agent, such as EDC, may be added to the mixture. The reaction mixture may be incubated. The crude 5-ALA-QD nanoparticle conjugate may be subject to purification to obtain the 5-ALA-QD nanoparticle conjugate.

Standard solid state purification method may be used. Several cycles of filtering and washing with a suitable solvent may be necessary to remove excess unreacted 5-ALA and EDC.

In another embodiment, the 5-ALA-nanoparticle conjugate may further include a ligand capable of targeting a cancer cell. For example, a chemical compound or a peptide, such as, for example, an antibody may be conjugated to the 5-ALA-nanoparticle conjugate to further effect cellular uptake of the 5-ALA-nanoparticle conjugate for either photo-detection or phototherapy. An example of a peptide is PLZ4 (QDGRMGF), which is a peptide that may selectively bind to bladder cancer cells. The peptides may form amide or ester bonds with the functionalized nanoparticle by their amine or carboxylic acid groups.

Once selectively bound to the cancer cell, the 5-ALA-nanoparticle conjugate will be taken up by the cell. Once internalized, 5-ALA undergoes conversion to the natural photosensitizer photoporphyrin IX (PpIX). Subsequent illumination of the tumor site with light, for example, blue light in the range of 375-475 nm, activates PpIX, triggers the oxidative damage with the release of reactive oxygen species (ROS) and induces cytotoxicity or apoptosis.

Accordingly, embodiments disclosed herein may be used for methods of inducing apoptosis of a cell, for example, a mammalian cell, comprising the step of administering a 5-ALA-nanoparticle conjugate to a mammal in need thereof, allowing 5-ALA to form metabolites, such as PpIX, and irradiating the metabolites. The irradiating step may be done by excitation of a nanoparticle, such as a disassociated nanoparticle.

Embodiments also include methods of detecting cancer cells by imaging the mammal.

The administration of the 5-ALA-nanoparticle conjugate may be enteral or parenteral. For example, the 5-ALA-nanoparticle conjugate may be administered subcutaneously, intravenously, intramuscular, topically, and orally. Examples include bolus injections or IV infusions.

The 5-ALA-QD nanoparticle conjugate of the current invention has the following advantages over the free 5-ALA.

First, the 5-ALA-QD nanoparticle conjugate has enhanced cell permeability and may be taken up more efficiently by the cancer cells, especially by the very active cancer stem cells. Nanoparticles in general accumulate in cancer cells more than normal cells. The QD nanoparticles act as a vectorized delivery system.

Second, the QD emission may be tuned to overlap with PpIX absorption. Once the QD-5ALA particles are internalized into the cancer cell, the 5-ALA will be released and transformed into PpIX within a few hours. The QDs then may be used as a light or FRET donor to enhance the excitation of the produced PpIX. Because QD nanoparticles have 10-100 fold higher molecular extinction coefficient compared to small molecular dyes like PpIXs, more light may be absorbed, and a stronger signal may be generated, improving signal to noise detection ratio.

Third, the high light absorption intensity may also increase the efficacy of PpIX in generating singlet oxygen as a photodynamic therapeutic (PDT) agent.

Fourth, the tunability of the QD nanoparticles and the potential for multi-photon excitation (including two-photo excitation) may enable deeper tissue detection and deeper PDT, unlike 5-ALA alone where only a few millimeters of tissue depth may be accessed.

Fifth, two-photo excitation or multiphoton excitation provides a means for excitation wavelength at greater than 700 nm, and allows PDT with highly localized light dosage.

These and other advantages of the present invention will be apparent to those skilled in the art from the foregoing specification. Accordingly, it is to be recognized by those skilled in the art that changes or modifications may be made to the above-described embodiments without departing from the broad inventive concepts of the invention. It is to be understood that this invention is not limited to the particular embodiments described herein, but is intended to include all changes and modifications that are within the scope and spirit of the invention.

What is claimed is:

1. A functionalized quantum dot nanoparticle conjugated to 5-aminolevulinic acids.

2. The 5-ALA-nanoparticle conjugate of claim 1, wherein the nanoparticle is covalently linked to 5-ALA via an amide or an ester bond.

3. The functionalized quantum dot nanoparticle of claim 1, wherein the quantum dot nanoparticle is a core-shell nanoparticle.

4. The functionalized quantum dot nanoparticle of claim 1, further comprising a ligand capable of targeting a cancer cell.

5. The functionalized quantum dot nanoparticle of claim 1, wherein the ligand is PLZ4.

6. The functionalized quantum dot nanoparticle of claim 1, wherein the quantum dot nanoparticle is substantially cadmium free.

7. A method of preparing a 5-ALA-nanoparticle conjugate comprising the steps of:
providing a nanoparticle comprising a molecular cluster compound, a core semiconductor material, and an outer layer;
providing a coupling agent;
providing 5-ALA, 5-ALA derivatives, or 5-ALA analogs;
incubating the mixture to form crude 5-ALA-nanoparticle conjugate;
purifying the crude 5-ALA-nanoparticle conjugate; and
isolating the 5-ALA-nanoparticle conjugate.

8. The method of claim 7, wherein the coupling agent is 1-(3-dimethylaminopropyl)-3-ethylcarbodiimide hydrochloride.

9. The method of claim 7, further comprising the step of conjugating the 5-ALA-nanoparticle conjugate to a ligand capable of targeting a cancer cell.

10. A method of inducing apoptosis of a cell comprising the steps of:
administering a functionalized nanoparticle conjugated to a plurality of 5-aminolevulinic acids to a mammal in thereof;
allowing 5-aminolevulinic acids to form metabolites; and
irradiating the metabolites.

11. The method of claim 10, wherein the metabolite is photoporphyrin IX.

12. The method of claim 10, wherein the step of irradiating is performed by the nanoparticle.

13. The method of claim 12, wherein the nanoparticle emits light in the range of 375-475 nm.

14. The method of claim 10, wherein the step of irradiating is sufficient to produces reactive oxygen species.

15. The method of claim 10, wherein the functionalized nanoparticle further comprises a ligand capable of targeting a cancer cell.

16. The method of claim 15, further comprising the step of the ligand binding to a cancer cell.

17. A method of detecting cancer cells comprising the steps of:
administering a 5-ALA-nanoparticle conjugate in photodynamic diagnosis as a precursor of both a fluorescence label and a photosensitizer;
allowing disassociation of 5-ALA from the nanoparticle;
allowing 5-ALA to form PpIX;
exciting a disassociated nanoparticle to emit blue light of 375-475 nm;
activating the fluorescent properties of PpIX; and
imaging the fluorescence.

18. The method of claim 17, wherein the administering step is performed by injection.

19. The method of claim 18, wherein the injection is performed intravenously.

20. The method of claim 19, wherein the nanoparticle is an alloyed quantum dot.
